(19) United States
(12) Patent Application Publication
(10) Pub. No.: US 2005/0054557 A1
(43) Pub. Date: Mar. 10, 2005

(54) Compositions for delivering parathyroid hormone and calcitonin

(76) Inventor: Michael M. Goldberg, Englewood, NJ (US)

Correspondence Address: DARBY & DARBY P.C., P. O. BOX 5257, NEW YORK, NY, 10150-5257, US

(21) Appl. No.: 10/787,857

(22) Filed: Feb. 25, 2004

Related U.S. Application Data
(63) Continuation of application No. 10/435,514, filed on May 9, 2003, now abandoned.
(60) Provisional application No. 60/379,501, filed on May 9, 2002.

(51) Int. Cl. A61K 38/29

(52) U.S. Cl. 514/2

(57) ABSTRACT

The present invention relates to a composition comprising a delivery agent, parathyroid hormone, and calcitonin. This composition exhibits increased delivery of parathyroid hormone and/or calcitonin and is useful for the treatment of osteoporosis. The composition also permits simultaneous oral delivery of parathyroid hormone and calcitonin. The composition of the present invention may be formulated into a dosage unit form, such as an oral dosage unit form. The invention also provides a method for administering parathyroid hormone and calcitonin to an animal in need thereof by administering the composition of the present invention.

This application claims the benefit of U.S. Provisional Application No. 60/379,501, filed May 9, 2002, which is hereby incorporated by reference.

### FIELD OF THE INVENTION

The present invention relates to a composition containing a delivery agent, parathyroid hormone, and calcitonin and a method of treating osteoporosis by administering the same to a patient in need thereof.

### BACKGROUND OF THE INVENTION

Calcitonin, which is known to aide in the prevention of bone loss, is frequently prescribed for the treatment of osteoporosis. Also, parathyroid hormone is known to treat osteoporosis. Unlike calcitonin, parathyroid hormone aides in bone regeneration.

There is a need for improved pharmaceutical formulations which are effective against osteoporosis.

### SUMMARY OF THE INVENTION

The present invention relates to a composition comprising a delivery agent, parathyroid hormone, and calcitonin. This composition exhibits increased delivery of parathyroid hormone and/or calcitonin and is useful for the treatment of osteoporosis. The composition also permits simultaneous oral delivery of parathyroid hormone and calcitonin. The composition of the present invention may be formulated into a dosage unit form, such as an oral dosage unit form.

The invention also provides a method for administering parathyroid hormone (PTH) and calcitonin to an animal in need thereof by administering the composition of the present invention. Accordingly, the present invention is directed to a method for orally administering an effective dose of PTH comprising orally co-administering to a patient in need of PTH an effective amount of a PTH and an effective amount of a calcitonin.

Administration of PTH to primates results in increased plasma concentrations of serum parathyroid hormone and serum calcium. Conversely the administration of salmon calcitonin (sCT) to primates results is an increase in serum sCT concentrations and a reduction in serum calcium. It has now been found that the oral administration of a combination of PTH and calcitonin, while resulting in similar PTH and calcitonin plasma concentration levels as those attained upon administrations of each agent alone; quite surprisingly results in reduction of serum calcium concentrations to the level observed with calcitonin alone. In effect, the calcitonin negates the hypercalcemic effect of the PTH while attaining the same reduction in serum calcium obtained when calcitonin is administered alone, in the absence of PTH. Administering calcitonin with PTH therapy allows the additional therapeutic effects of the presently precluded PTH doses without the hypercalcemic side effects. Additionally, the calcitonin provides an analgesic effect which is useful in off-setting the bone pain usually associated with administration of PTH.

The invention is also directed to a method of stimulating new bone formation comprising orally administering to a patient in need of new bone formation a therapeutically effective amount of a PTH and a therapeutically effective amount of a calcitonin. In a further embodiment, the invention is directed to a method of treatment or prevention of osteoporosis comprising orally administering to a patient in need of said treatment or prevention a therapeutical effective amount of a PTH and a therapeutically effective amount of a calcitonin.

The invention is also directed to a composition suitable for oral delivery comprising a PTH and a calcitonin, e.g. for simultaneous, concurrent or sequential administration of the PTH and calcitonin.

The invention is further directed to use of PTH and calcitonin for the preparation of an orally administrable medicament for the stimulation of new bone formation, e.g. for simultaneous, concurrent or sequential oral administration of the PTH and calcitonin.

The invention is yet further directed to a kit for the stimulation of new bone formation comprising PTH and calcitonin suitable for oral administration together with instructions for the oral administration thereof, e.g. for simultaneous, concurrent or sequential oral administration of the PTH and calcitonin.

The present invention is further directed to pharmaceutical compositions suitable for oral delivery of PTH fragments and to methods of administering such compositions. Specifically, the instant invention is directed to a pharmaceutical composition for oral delivery comprising a therapeutically effective amount of a PTH fragment and 5-CNAC, said PTH fragment selected from PTH (1-28) to PTH (1-41). Preferably, the PTH is human parathyroid hormone, hPTH.

In another embodiment, the invention is directed to a method for orally administering an effective dose of PTH comprising orally administering to a patient in need of PTH a pharmaceutical composition comprising a therapeutically effective amount of a PTH fragment and 5-CNAC, said PTH fragment selected from PTH (1-28) to PTH (1-41).

The invention is also directed to a method of stimulating new bone formation comprising orally administering to a patient in need of new bone formation a pharmaceutical composition comprising a therapeutically effective amount of a PTH fragment and 5-CNAC, said PTH fragment selected from PTH (1-28) to PTH (1-41).

In a further embodiment, the invention is directed to a method of treatment or prevention of osteoporosis comprising orally administering to a patient in need of said treatment or prevention a pharmaceutical composition comprising a therapeutically effective amount of a PTH fragment and 5-CNAC, said PTH fragment selected from PTH (1-28) to PTH (1-41).

In a still further embodiment, the invention is directed to the use of 5-CNAC for the preparation of a pharmaceutical composition suitable for the oral delivery of PTH fragments selected from PTH (1-28) to PTH (1-41).

International Publication Nos. WO 02/098453 and WO 03/015822 are hereby incorporated by reference.

### DETAILED DESCRIPTION OF THE INVENTION

Delivery Agents

Suitable delivery agents include, but are not limited to, those having the formula 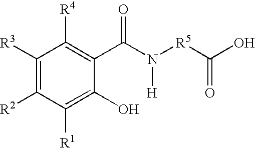
wherein
R¹, R², R³, and R⁴ are independently hydrogen, —OH, —NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C₁₂ alkylene); and

R⁶ and R⁷ are independently hydrogen, oxygen, or C₁-C₄ alkyl,

or a salt thereof, solvate thereof, or hydrate thereof. Preferred delivery agents include, but are not limited to, N-(5-chlorosalicyloyl)-8-aminocaprylic acid (5-CNAC) and salts, solvates, and hydrates thereof. Other non-limiting examples of delivery agents are N-(10-[2-hydroxybenzoyl]-amino)decanoic acid (SNAD) and N-(8-[2-hydroxybenzoyl]amino)caprylic acid (SNAC). The compounds above as well as their monosodum and disodium salts and alcohol solvates and hydrates thereof are described in WO 00/59863 (which is hereby incorporated by reference), along with method for preparing them.

The preferred delivery agents can be 5-CNAC, SNAC, SNAD, and their monosodium and disodium salts, ethanol solvates of their sodium salts and the monohydrates of their sodium salts and any combinations thereof. The most preferred delivery agent is the disodium salt of 5-CNAC and the monohydrate thereof.

Other suitable delivery agents are any one of the 123 modified amino acids disclosed in U.S. Pat. No. 5,866,536 or any one of the 193 modified amino acids described in U.S. Pat. No. 5,773,647 or any combination thereof. The contents of the aforementioned U.S. Pat. Nos. 5,773,647 and 5,866,536 are hereby incorporated by reference in their entirety.

For purposes of the instant invention, the 5-CNAC, i.e. N-(5-chlorosalicyloyl)-8-aminocaprylic acid, can be the free acid, analogs thereof, its monosodium and disodium salts, ethanol solvates of the sodium salts and the monohydrates of the sodium salts and any combinations thereof. The free acid, the disodium salt of 5-CNAC and the monohydrate thereof are particularly useful. N-(5-chlorosalicyloyl)-8-aminocaprylic acid is described in the aforementioned '647 patent, the contents of which are hereby incorporated by reference, and can be made by methods described therein. The sodium salts and alcohol solvates and hydrates thereof are described in WO 00/059,863, along with methods for preparing them.

The disodium salt may be prepared from the ethanol solvate by evaporating or drying the ethanol solvate by methods known in the art to form the anhydrous disodium salt. Drying is generally carried out at a temperature of from about 80 to about 120 C, preferably from about 85 to about 90 C, and most preferably at about 85 C. The drying step is generally performed at a pressure of 26″Hg or greater. The anhydrous disodium salt generally contains less than about 5% by weight of ethanol and preferably less than about 2% by weight of ethanol, based on 100% total weight of anhydrous disodium salt.

The disodium salt of N-(5-chlorosalicyloyl)-8-aminocaprylic acid can also be prepared by making a slurry of N-(5-chlorosalicyloyl)-8-aminocaprylic acid in water and adding two molar equivalents of aqueous sodium hydroxide, sodium alkoxide or the like. Suitable sodium alkoxide include, but are not limited to, sodium methoxide, sodium ethoxide, and combinations thereof.

A still further method of preparing the disodium salt is by reacting N-(5-chlorosalicyloyl)-8-aminocaprylic acid with one molar equivalent of sodium hydroxide to form a monosodium salt and then adding an additional one molar equivalent of sodium hydroxide to yield the disodium salt.

The disodium salt can be isolated as a solid by concentrating the solution containing the disodium salt to a thick paste by vacuum distillation. This paste may be dried in a vacuum oven to obtain the disodium salt of N-(5-chlorosalicyloyl)-8-aminocaprylic acid as a solid. The solid can also be isolated by spray drying an aqueous solution of the disodium salt.

The ethanol solvates, as described in the aforementioned WO 00/059,863, include, but are not limited to, a molecular or ionic complex of molecules or ions of ethanol solvent with molecules or ions of the disodium salt of N-(5-chlorosalicyloyl)-8-aminocaprylic acid. Typically, the ethanol solvate contains about one ethanol molecule or ion for every molecule of disodium salt of N-(5-chlorosalicyloyl)-8-aminocaprylic acid.

The ethanol solvate of the disodium salt of N-(5-chlorosalicyloyl)-8-aminocaprylic acid can be prepared by dissolving N-(5-chlorosalicyloyl)-8-aminocaprylic acid in ethanol. Typically, each gram of N-(5-chlorosalicyloyl)-8-aminocaprylic acid is dissolved in from about 1 to about 50 mL of ethanol and generally, from about 2 to about 10 mL of ethanol. The N-(5-chlorosalicyloyl)-8-aminocaprylic acid/ethanol solution is then reacted with a molar excess of a sodium containing salt, such as a monosodium containing salt, relative to N-(5-chlorosalicyloyl)-8-aminocaprylic acid, i.e. for every mole of N-(5-chlorosalicyloyl)-8-aminocaprylic acid there is more than one mole of sodium cations, yielding the ethanol solvate. Suitable monosodium salts include, but are not limited to, sodium hydroxide; sodium alkoxide, such as sodium methoxide and sodium ethoxide; and any combination of the foregoing. Preferably, at least about two molar equivalents of the monosodium containing salt are added to the ethanol solution, i.e. for every mole of N-(5-chlorosalicyloyl)-8-aminocaprylic acid there is at least about two moles of sodium cations.

Generally, the reaction is performed at or below the reflux temperature of the mixture, such as at ambient temperature. The ethanol solvate is then recovered by methods known is the art, such as, concentration of the resulting slurry at atmospheric distillation, cooling the concentrated slurry and filtering the solid. The recovered solid can then be vacuum dried to obtain the ethanol solvate.

The hydrates of the disodium salts of the N-(5-chlorosalicyloyl)-8-aminocaprylic acid may be prepared by drying the ethanol solvate to form an anhydrous disodium salt, as described above, and hydrating the anhydrous disodium salt. Preferably, the monohydrate of the disodium salt is formed. Since the anhydrous disodium salt is very hydroscopic, the hydrate forms upon exposure to atmospheric moisture. Generally, the hydrating step is performed at from about ambient temperature to about 50 C, preferably ambient temperature to about 30 C and in an environment having at least 50% relative humidity. Alternatively, the anhydrous disodium salt may be hydrated with steam.

Parathyroid Hormone

Any form of parathyroid hormone known in the art may be used. Suitable forms include, but are not limited to, human parathyroid hormone, such as human parathyroid hormone residues 1-34 (e.g. pTH-(1-34) or cyclical hpTH-(1-34)). The parathyroid hormone or PTH can be the full length, 84 amino acid form of parathyroid hormone, e.g. the human form, hPTH (1-84), or any polypeptide, protein, protein fragment, or modified fragment, i.e. PTH-related peptides and PTH analogs, capable of mimicing the activity of hPTH (1-84) in controlling calcium and phosphate metabolism to build bone in the human body. The PTH fragments will generally incorporate at least the first 28 N-terminal residue and include PTH (1-28), PTH (1-31), PTH (1-34), PTH (1-37), PTH (1-38) and PTH (1-41) or analogues thereof, e.g. PTS893. The PTH can be a single PTH or any combination of two or more PTHs. These parathyroid hormones are commercially available or can be obtained recombinantly, by peptide synthesis, or by extraction from human fluid by methods well established in the art.

The PTH fragments can be of any parathyroid hormone, particularly mammalian parathyroid hormone, e.g. human (hPTH), bovine (bPTH), and porcine (pPTH) and particularly hPTH and will incorporate at least the first 28 N-terminal residues (PTH (1-28)) up to and including the first 41 N-terminal residues (PTH (1-41)) and include without limitation PTH (1-28), PTH (1-31), PTH (1-34), PTH (1-37), PTH (1-38) and PTH (1-41). Human parathyroid hormone (1-34) is particularly preferred. These parathyroid hormone fragments are commercially available or can be obtained recombinantly or by peptide synthesis.

Calcitonin

Suitable forms of calcitonin include, but are not limited to, salmon calcitonin, eel calcitonin, human calcitonin, porcine calcitonin, and any combination of any of the foregoing. The calcitonin for use in the instant invention can be any calcitonin, including natural, synthetic or recombinant sources thereof, as well as calcitonin derivatives such as 1,7-Asn-eel calcitonin. Various calcitonins, including salmon, pig and eel calcitonin are commercially available and commonly employed for the treatment of e.g. Paget's disease, hypercalcemia of malignancy and osteoporosis. The calcitonin can comprise a single calcitonin or any combination of two or more calcitonins. The preferred calcitonin is synthetic salmon calcitonin. The calcitonins are commercially available or may be obtained by known methods.

Delivery Agent/Parathyroid Hormone/Calcitonin Composition

The amount of parathyroid hormone and calcitonin in the composition is an amount effective to accomplish the purpose intended. The amount in the composition is typically a pharmacologically, biologically, therapeutically, or chemically effective amount. However, the amount can be less than that amount when a plurality of the compositions are to be administered, i.e., the total effective amount can be administered in cumulative units. The amount of parathyroid hormone and calcitonin can also be more than a pharmacologically, biologically, therapeutically, or chemically effective amount when the composition provides sustained release of the active agent. Such a composition typically has a sustained release coating which causes the composition to release a pharmacologically, biologically, therapeutically, or chemically effective amount of the active agent over a prolonged period of time.

The amount of PTH to be administered is generally an amount effective to stimulate new bone formation i.e. a therapeutically effective amount. This amount will necessarily vary with the age, size, sex and condition of the subject to be treated, the nature and severity of the disorder to be treated and the like. However, the amount can be less than that amount when a plurality of the compositions are to be administered, i.e., the total effective amount can be administered in cumulative dosage units. The amount of PTH can also be more than the effective amount when the composition provides sustained release of the pharmacologically active agent. The total amount of PTH to be used can be determined by methods known to those skilled in the art. However, in general, satisfactory results will be obtained systemically at daily dosages of from about 0.001 ug/kg to about 10 mg/kg animal body weight, preferably 1 ug/kg to about 6 ug/kg body weight.

The appropriate dosage of calcitonin to be administered will, of course, vary depending upon, for example, the amount of PTH to be administered and the severity of the condition being treated. However, in general, satisfactory results will be obtained systemically at daily dosages of from about 0.5 Rg/kg to about 10 ug/kg animal body weight, preferably 1 llg/kg to about 6 ug/kg body weight.

The total amount of active parathyroid hormone and calcitonin to be used can be determined by methods known to those skilled in the art. However, because the compositions may deliver these active agents more efficiently than prior compositions, lesser amounts of the active agents than those used in prior dosage unit forms or delivery systems can be administered to the subject, while still achieving the same blood levels and/or therapeutic effects.

Generally, the weight ratios of delivery agent to calcitonin and delivery agent to parathyroid hormone varies depending on the animal to which the composition is to be administered. For example, for a composition which is to be administered to humans the weight ratio of delivery agent to calcitonin in the composition may range from about 100:1 to about 1000:1, preferably is from about 400:1 to about 600:1, and is most preferably about 500:1. The weight ratio of delivery agent to parathyroid hormone in the composition may range from about 100:1 to about 1000:1, preferably is from about 400:1 to about 600:1, and is most preferably about 500:1.

The pharmaceutical compositions of the present invention typically contain a delivery effective amount of one or more of the delivery agents, i.e. an amount sufficient to deliver the PTH and/or calcitonin for the desired effect. Generally, the delivery agent is present in an amount of 2.5% to 99.4% by weight, more preferably 25% to 50% by weight of the total composition.

The composition may further comprise additives known in the art. Suitable additives include, but are not limited to, pH adjusters, preservatives, flavorants, taste-masking agents, fragrances, humectants, tonicifiers, colorants, surfactants, plasticizers, lubricants (such as magnesium stearate), flow aids, compression aids, dosing vehicles, solubilizers, excipients, diluents (such as microcrystalline cellulose, e.g., Avicel PH 102 supplied by FMC corporation), disintegrants, and any combination of any of the foregoing. Suitable dosing vehicles include, but are not limited to, water, phosphate buffer, 1,2-propane diol, ethanol, olive oil, and any combination of any of the foregoing. Other additives include phosphate buffer salts, citric acid, glycols, and other dispersing agents. Stabilizing additives may be incorporated into the solution, preferably at a concentration ranging between about 0.1 and 20% (w/v). The composition may also include one or more enzyme inhibitors, such as actinonin or epiactinonin and derivatives thereof; aprotinin, Trasylol and Bowman-Birk inhibitor. Further, a transport inhibitor, i.e. a p-glycoprotein such as Ketoprofin, may be present in the compositions of the present invention.

The oral administration can be accomplished regularly, e.g. once or more on a daily or weekly basis; intermittently, e.g. irregularly during a day or week; or cyciically, e.g. regularly for a period of days or weeks followed by a period without administration.

The co-administration of PTH and calcitonin includes simultaneous, concurrent, or sequential administration of the two compounds. Simultaneous administration means administration of the two compounds in a single dosage form; concurrent administration means administration of the two compounds at about the same time but in separate dosage forms; and, sequential administration means administration of one of the compounds, after which the other is administered. Sequential administration may also take the form of simultaneous or concurrent administration of the two compounds, followed by cessation of the simultaneous or concurrent administration and then continued administration of one of the two compounds alone.

The composition may be formulated into a dosage unit form (e.g., a liquid or solid dosage form), such as a tablet, capsule, powder, or liquid. The liquid dosage forms include solution emulsions, suspensions, syrups and elixirs. In addition to the PTH and/or calcitonin, the liquid formulations may also include inert excipients commonly used in the art such as, solubilizing agents such as ethanol; oils such as cottonseed, castor and sesame oils; wetting agents; emulsifying agents; suspending agents; sweeteners; flavorings; and solvent such as water. The solid dosage forms include capsules, soft-gel capsules, tablets, caplets, powders, granules or other solid oral dosage forms, all of which can be prepared by methods well known in the art. In addition to the PTH and/or calcitonin, these solid dosage forms generally include a pharmaceutical acceptable delivery agent for the PTH and/or calcitonin.

The composition may be prepared by dry mixing or mixing in solution the delivery agent, parathyroid hormone, calcitonin, and, optionally, additives. The mixture may be gently heated and/or inverted to aid in dispersing the components in solution.

The solid pharmaceutical compositions of the instant invention can be prepared by conventional methods e.g. by blending a mixture of the active agent or active agents, the delivery agent, and any other ingredients, kneading, and filling into capsules or, instead of filling into capsules, molding followed by further tableting or compression-molding to give tablets. In addition, a solid dispersion may be formed by known methods followed by further processing to form a tablet or capsule.

Preferably, the ingredients in the pharmaceutical compositions of the instant invention are homogeneously or uniformly mixed throughout the solid dosage form.

The composition and dosage unit form of the present invention may be administered to an animal in need thereof, including but not limited to, birds, such as chickens; mammals, such as rodents, cows, pigs, dogs, cats, primates, and particularly humans; and insects. The composition and dosage unit form may be administered by the oral, intranasal, sublingual, intraduodenal, subcutaneous, buccal, intracolonic, rectal, vaginal, mucosal, pulmonary, transdermal, intradermal, parenteral, intravenous, intramuscular or ocular route. Preferably, the composition and dosage unit form are administered orally.

The composition and dosage unit form may be administered to treat osteoporosis. The amount of the delivery agent, parathyroid hormone, and calcitonin administered is an amount effective for treating osteoporosis.

5-CNAC/Parathyroid Hormone

The description above regarding the delivery agents and parathyroid hormone above also applies to this composition.

The amount of PTH fragment to be administered is generally an amount effective to stimulate new bone formation i.e. a therapeutically effective amount. This amount will necessarily vary with the age, size, sex and condition of the subject to be treated, the nature and severity of the disorder to be treated and the like. However, the amount can be less than that amount when a plurality of the compositions are to be administered, i.e., the total effective amount can be administered in cumulative dosage units. The amount of PTH can also be more than the effective amount when the composition provides sustained release of the pharmacologically active agent.

The total amount of PTH to be used can be determined by methods known to those skilled in the art. However, in general, satisfactory results will be obtained systemically at daily dosages of from about 0.001 Fg/kg to about 10 mg/kg animal body weight, preferably 1 ug/kg to about 6 ug/kg body weight.

The pharmaceutical compositions of the present invention typically contain a delivery effective amount of 5-CNAC, i.e. an amount sufficient to deliver the PTH for the desired effect.

Generally, the 5-CNAC is present in an amount of 2.5% to 99.4% by weight, more preferably 25% to 50% by weight of the total composition.

Oral administration of the pharmaceutical compositions according to the invention can be accomplished regularly, e.g. once or more on a daily or weekly basis; intermittently, e.g. irregularly during a day or week; or cyclically, e.g. regularly for a period of days or weeks followed by a period without administration.

The dosage form of the pharmaceutical compositions of the instant invention can be any known form, e.g. liquid or solid dosage forms.

The liquid dosage forms include solution emulsions, suspensions, syrups and elixirs. In addition to the PTH and 5-CNAC, the liquid formulations may also include inert excipients commonly used in the art such as, solubilizing agents e.g. ethanol; oils such as cottonseed, castor and sesame oils; wetting agents; emulsifying agents; suspending agents; sweeteners; flavorings; and solvents such as water.

The solid dosage forms include capsules, soft-gel capsules, tablets, caplets, powders, granules or other solid oral dosage forms, all of which can be prepared by methods well known in the art.

The pharmaceutical compositions may additionally comprise additives in amounts customarily employed including, but not limited to, a pH adjuster, a preservative, a flavorant, a taste-masking agent, a fragrance, a humectant, a tonicifier, a colorant, a surfactant, a plasticizer, a lubricant such as magnesium stearate, a flow aid, a compression aid, a solubilizer, an excipient, a diluent such as microcrystalline cellulose, e.g. Avicel PH 102 supplied by FMC corporation, or any combination thereof. Other additives may include phosphate buffer salts, citric acid, glycols, and other dispersing agents.

The composition may also include one or more enzyme inhibitors, such as actinonin or epiactinonin and derivatives thereof; aprotinin, Trasylol and Bowman-Birk inhibitor.

Further, a transport inhibitor, i.e. a p-glycoprotein such as Ketoprofin, may be present in the compositions of the present invention.

The solid pharmaceutical compositions of the instant invention can be prepared by conventional methods e.g. by blending a mixture of the PTH fragment, the 5-CNAC, and any other ingredients, kneading, and filling into capsules or, instead of filling into capsules, molding followed by further tableting or compression-molding to give tablets. In addition, a solid dispersion may be formed by known methods followed by further processing to form a tablet or capsule.

Preferably, the ingredients in the pharmaceutical compositions of the instant invention are homogeneously or uniformly mixed throughout the solid dosage form.

Parathyroid hormones are indicated for preventing or treating all bone conditions which are associated with increased calcium depletion or resorption or in which stimulation of bone formation and calcium fixation in the bone is desirable, e.g. osteoporosis of various genesis (e.g. juvenile, menopausal, post-menopausal, post-traumatic, caused by old age or by corticoid-steroid therapy or inactivity), fractures, osteopathy, including acute and chronic states associated with skeletal demineralization, osteo-malacia, periodontal bone loss or bone loss due to arthritis or osteoarthritis or cancer (e.g. bone metastis) or for treating hypoparathyroidism.

Parathyroid hormones are particularly indicated for preventing or treating osteoporosis of various genesis.

According to a further embodiment of the invention, the PTH may be employed as adjunct or adjuvant to other therapy, e.g. a therapy using a bone resorption inhibitor, for example as in osteoporosis therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, TSE-424, FC1271, Tibolone (Livial &commat;), vitamin D or an analogue thereof or an activator of PTH release, or bisphosphonates, e.g. clodronic acid, etidronic acid, pamidronic acid, aledronic acid, ibandronic acid, zoledronic acid, risedronic acid or tiludronic acid and salts and hydrates thereof.

When the PTH is administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth.

The oral administration of the present invention may be to any animal in need thereof, including, but not limited to, mammals, such as rodents, cows, pigs, dogs, cats, and primates, particularly humans.

The following examples are intended to describe the present invention without limitation.

### EXAMPLE 1

### Preparation of N-(5-chlorosalicyloyl)-8-aminocaprylic acid (5-CNAC)

To a clean, dry, 200 gallon glass-lined reactor, 178 L of dry acetonitrile was added. The agitator was set to 100-125 rpm and the reactor contents were cooled to about 9° C. 74 kg of 5-chloro salicylamide, available from Polycarbon Industries of Leominster, Mass., was charged to the reactor and the charging port was closed. 47 L of dry pyridine was charged to the reactor. The resulting slurry was cooled to about 9° C. Cooling was applied to the reactor condenser and valve overheads were set for total reflux. Over 2 hours, 49.7 kg of ethylchloroformate was charged to the 200 gallon reactor while maintaining the batch temperature at about 14° C. Ethylchloroformate can contain 0.1% phosgene and is extremely reactive with water. The reaction is highly exothermic and requires the use of a process chiller to moderate reaction temperature.

The reactor contents were agitated for about 30 minutes at 10-14° C., once the ethylchloroformate addition was complete. The reactor contents were then heated to about 85° C. over about 25 minutes, collecting all distillate into a receiver. The reactor contents were held at 85-94° C. for approximately 6 hours, collecting all distilled material into a receiver. The reaction mixture was sampled and the conversion (>90%) monitored by HPLC. The conversion was found to be 99.9% after 6 hours. The reactor contents were cooled to about 19° C. over a one-hour period. 134 L of deionized water was charged to the reactor. A precipitate formed immediately. The reactor contents were cooled to about 5° C. and agitated for about 10.5 hours. The product continued to crystallize out of solution. The reactor slurry was centrifuged. 55 L of deionized water was charged to the 200-gallon, glass-lined reactor and the centrifuge wet cake was washed. The intermediate was dried under full vacuum (28″ Hg) at about 58° C. for about 19.5 hours. The yield was 82.6 kg 6-chloro-2H-1,3-benzoxazine-2,4(3H)-dione. This intermediate was packaged and stored so that it was not exposed to water.

In the following preparation, absolutely no water can be tolerated in the steps up to the point where distilled water is added. 222 L of dry dimethylacetamide was charged to a dry 200 gallon glass-lined reactor. The reactor agitator was set to 100-125 rpm. Cooling was applied to the condenser and valve reactor overheads were set for distillation. 41.6 kg of dry anhydrous sodium carbonate was charged to the reactor and the reactor charging port was closed. Caution was used due to some off-gassing and a slight exothermic reaction. 77.5 kg of dry 6-chloro-2H-1,3-benzoxazine-2,4(3H)-dione was charged to the reactor. Quickly, 88 kg of dry ethyl-8-bromooctanoate was charged to the reactor. The reaction was evacuated to 22-24 inches of vacuum and the reactor temperature was raised to 65-75° C. The reactor temperature was maintained and the contents were watched for foaming. The reactor mixture was sampled and monitored for conversion by monitoring the disappearance of the bromo ester in the reaction mixture by gas chromatography. The reaction was complete (0.6% bromo ester was found) after about 7 hours. The vacuum was broken and the reactor contents were cooled to 45-50° C. The contents were centrifuged and the filtrate sent into a second 200 gallon glass-lined reactor. 119 L of ethanol (200 proof denatured with 0.5% toluene) was charged to the first 200 gallon reactor, warmed to about 45° C. The filter cake was washed with warm ethanol and the wash was charged to the reaction mixture in the second 200 gallon reactor.

The agitator was started on the second 200 gallon reactor. The reactor contents were cooled to about 29° C. 120 L distilled water was slowly charged to the second reactor, with the water falling directly into the batch. The reactor contents were cooled to about 8° C. The intermediate came out of solution and was held for about 9.5 hours. The resultant slurry was centrifuged. 70 L ethanol was charged to the reactor, cooled to about 8° C., and the centrifuge cake was washed. The wet cake was unloaded into double polyethylene bags placed inside a paper lined drum. The yield was 123.5 kg of ethyl 8-(6-chloro-2H-1,3-benzoxazine-2,4(3H)-dionyl)octanoate.

400 L purified water, USP and 45.4 kg sodium hydroxide pellets were charged to a 200 gallon glass-lined reactor and the agitator was set to 100-125 rpm. 123.5 kg of the ethyl 8-(6-chloro-2H-1,3-benzoxazine-2,4(3H)-dionyl)octanoate wet cake was charged to the reactor. The charging port was closed. Cooling water was applied to the condenser and the valve reactor overheads were set for atmospheric distillation. The reactor contents were heated to about 98° C. and the conversion was monitored by HLPC. Initially (approximately 40 minutes) the reactor refluxed at about 68° C., however, as the ethanol was removed (over about 3 hours) by distillation the reactor temperature rose to about 98° C. The starting material disappeared, as determined by HPLC, at approximately 4 hours. The reactor contents were cooled to about 27° C. 150 L purified water, USP was charged to an adjacent 200 gallon glass-lined reactor and the agitator was set to 100-125 rpm. 104 L concentrated (12M) hydrochloric acid was charged to the reactor and cooled to about 24° C. The saponified reaction mixture was slowly charged (over about 5 hours) to the 200 gallon glass-lined reactor. The material (45 L and 45 L) was split into 2 reactors (200 gallons each) because of carbon dioxide evolution. The product precipitated out of solution. The reaction mixture was adjusted to pH 2.0-4.0 with a 50% sodium hydroxide solution (2L water, 2 kg sodium hydroxide). The reactor contents were cooled to about 9-15° C. The intermediate crystallized out of solution over approximately 9 hours. The reactor slurry was centrifuged to isolate the intermediate. 50 L purified water, USP was charged to a 200 gallon glass-lined reactor and this rinse was used to wash the centrifuge wet cake. The wet cake was unloaded into double polyethylene bags placed inside a plastic drum. The N-(5-chlorosalicyloyl)-8-aminocaprylic acid was dried under vacuum (27″ Hg) at about 68° C. for about 38 hours. The dry cake was unloaded into double polyethylene bags placed inside a 55-gallon, steel unlined, open-head drums with a desiccant bag placed on top. The dried isolated yield was 81 kg of N-(5-chlorosalicyloyl)-8-aminocaprylic acid.

### EXAMPLE 2

### Preparation of Disodium N-(5-chlorosalicyloyl)-8-aminocaprylate Ethanol Solvate

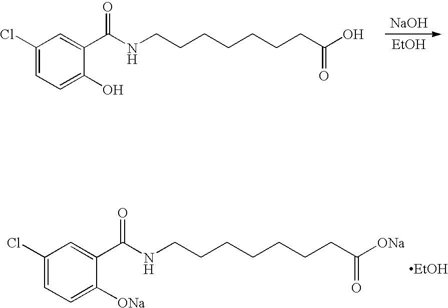

A 12 L, Pyrex glass, four-neck, round bottom flask was equipped with an overhead stirrer, thermocouple temperature read out, reflux condenser, and heating mantle. The flask was purged with dry nitrogen and the following reaction was conducted under an atmosphere of dry nitrogen. The flask was charged with 1000 g of N-(5-chloro-salicyloyl)-8-aminooctanic acid and 3000 mL of absolute ethanol. This slurry was heated to 55° C. with stirring to obtain a slightly hazy solution. The reactor was then charged with 2276 g of 11.2 wt % sodium hydroxide dissolved in absolute ethanol as rapidly as possible. There was a slight exothermic reaction causing the temperature in the reactor to rise to about 64° C. and a precipitate began to form. The reflux condenser was removed and the reactor set for distillation. The reaction mixture was distilled over the next three hours to obtain about 2566 g of distillate. The pot slurry was allowed to cool slowly to room temperature. The product solids in the slurry were recovered by vacuum filtration through a sintered glass funnel to obtain 1390 g of ethanol wet cake. The wet cake was transferred to glass trays and placed in a vacuum oven. The cake was dried to constant weight at about 45° C. and full vacuum. The dry product had a weight of about 1094.7 g.

Titration of the product with hydrochloric acid gave two inflection points consuming approximately 2 molar equivalents of hydrochloric acid. CHN analysis: theoretical (correcting 0 wt % water) C 50.56%, H 5.99%, N 3.47%, Na 11.39%; actual C 50.24%, H 5.74%, N 3.50% (Na was not measured).

### EXAMPLE 3

### Preparation of Disodium N-(5-chlorosalicyloyl)-8-aminocaprylate

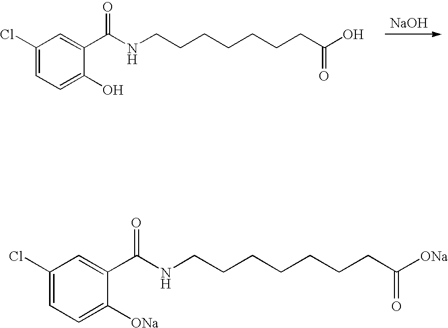

A 22 L, Pyrex glass, five-neck, round bottom flask was equipped with an overhead stirrer, thermocouple temperature read out, and heating mantle. The flask was charged with 2602.3 g of N-(5-chlorosalicyloyl)-8-aminocaprylic acid and 4000 mL water. To this stirred slurry was added a solution of 660 g of sodium hydroxide dissolved in 2000 mL water. The mixture was heated to about 55° C. and most of the solids dissolved. The slightly hazy solution was hot filtered through Whatman #1 filter paper to remove the insoluble particulates. The filtrate was transferred to the pot flask of a large laboratory rotary evaporator. The rotary evaporator was operated with a bath temperature of about 60° C. and a pressure of 60 mmHg. Water was removed from the disodium salt solution until a solid mass was obtained in the rotary evaporator pot flask. The vacuum was released and pot flask removed from the rotary evaporator. The solids were scraped from the pot flask into trays. These trays were then placed in a vacuum oven and the solids dried at about 60° C. and full vacuum for about 48 hours. The dried solids were run through a laboratory mill until all the solids passed through a 35 mesh screen. The milled and sieved disodium N-(5-chlorosalicyloyl)-8-aminooctanate was put into trays and placed back into the drying oven. Drying was continued at about 45° C. and full vacuum to obtain 2957.1 g of the desired product as a dry powder.

Titration of the product with hydrochloric acid gave two inflection points consuming approximately 2 molar equivalents of hydrochloric acid. CHN analysis: theoretical (correcting 4.9 wt % water) C 47.89%, H 5.37%, N 3.72%, Na 12.22%; actual C 47.69%, H 5.23%, N 3.45%, Na 11.79%.

### EXAMPLE 4

### Preparation of Monosodium N-(5-chlorosalicyloyl)-8-aminocaprylate

A 22 L, Pyrex glass, five-neck, round bottom flask was equipped with an overhead stirrer, thermocouple temperature read out, and heating mantle. The flask was charged with 2099.7 g of N-(5-chlorosalicyloyl)-8-aminooctanoic acid and 6000 mL water and stirred. To this slurry was added a solution of 265 g of sodium hydroxide dissolved in 2000 mL water. The mixture was heated to about 80° C. causing most of the solids to dissolve. The undissolved material was allowed to settle to the bottom of the flask and the supernate decanted. The resulting mixture was transferred to the pot flask of a large laboratory rotary evaporator. The rotary evaporator was operated with a bath temperature of about 60° C. and a pressure of about 70 mmHg. Water was removed from the disodium salt mixture until a solid mass was obtained in the rotary evaporator pot flask. The vacuum was released and pot flask removed from the rotary evaporator. The solids were scraped from the pot flask into trays. These trays were then placed in a vacuum oven and the solids dried at about 60° C. and full vacuum for about 48 hours. The dried solids were run through a laboratory mill until all the solids passed through a 35 mesh screen. The milled and seived disodium N-(5-chlorosalicyloyl)-8-aminooctanate was put into trays and placed back into the drying oven. Drying was continued at full vacuum to yield 2161.7 g of the desired product as a dry powder.

Titration of the product with hydrochloric acid gave a single inflection point consuming approximately 1 molar equivalent of hydrochloric acid. CHN analysis: theoretical (correcting 1.14 wt % water) C 53.05%, H 5.77%, N 4.12%, Na 6.77%; actual C 52.57%, H 5.56%, N 4.06%, Na 6.50%.

### EXAMPLE 5

### Preparation of Capsules Containing Disodium N-(5-chlorosalicyloyl)-8-aminocaprylate Ethanol Solvate, Parathyroid Hormone, and Salmon Calcitonin

5-CNAC disodium salt ethanol solvate was screened through a 35 mesh Tyler standard sieve. 13.54 g of this material was weighed out and kept in a weighing boat. 0.028 g of parathyroid hormone (PTH) and 0.025 g of salmon calcitonin (sCT) were separately weighed out and kept in separate weighing boats. An amount of the previously weighed 5-CNAC disodium salt ethanol solvate approximately equivalent in volume to the total volume of PTH (i.e. a geometric amount) was prescreened through the same 35 mesh Tyler standard sieve on to clean paper. All the PTH was then screened through the same sieve onto the same paper. An amount of the screened 5-CNAC disodium salt ethanol solvate equivalent in volume to the total amount of material on the paper was screened through the same sieve. This was followed by screening the sCT through the same sieve on to the paper. A geometric amount of the 5-CNAC disodium salt ethanol solvate was then screened through the same sieve onto the paper. The materials on the paper were then transferred to a glass mortar and mixed by light trituration with a pestle. The remainder of the 5-CNAC disodium salt ethanol solvate was screened through the same 35 mesh Tyler standard sieve. The 5-CNAC disodium salt ethanol solvate was transferred to the weighing boats that previously contained the sCT and PTH. A geometric amount of 5-CNAC disodium salt ethanol solvate was then added to the contents of the mortar and mixed thoroughly. The contents of the mortar were transferred to a 1 pint V-blender shell and mixed for 5 minutes. The material was then transferred to a weighing boat for manual capsule filling. The formulation was then hand filled into size 2 TORPAC® capsules, available from Torpac Inc. of West Fairfield, N.J. Forty capsules were made. The average capsule content weight was 227.15 mg. Each capsule contained approximately 226.28 mg 5-CNAC disodium salt ethanol solvate, 0.461 mg of PTH, and 0.411 mg of sCT.

Four cynomolgous monkeys were dosed via a feeding tube two capsules each followed by 10 ml water. The mean peak blood calcitonin concentration, Cₘₐₓ, of sCT was then measured.

The results showed that the mean peak blood concentration of sCT was about 2,000-2,500 pg/ml, which is about four times that found when dosing capsules of 5-CNAC and sCT in rhesus monkeys.

All patents, publications, applications, and test methods mentioned above are hereby incorporated by reference. Many variations of the present matter will suggest themselves to those skilled in the art in light of the above detailed description. All such obvious variations are within the patented scope of the appended claims.

1. A composition comprising a delivery agent, parathyroid hormone, and calcitonin.

2. The composition of claim 1, wherein the delivery agent has the formula 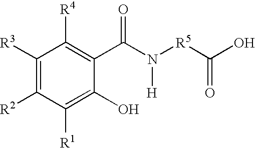
wherein
R¹, R², R³, and R⁴ are independently hydrogen, —OH, —NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C₁₂ alkylene); and
R⁶ and R⁷ are independently hydrogen, oxygen, or c₁-C₄ alkyl, or a salt thereof, solvate thereof, or hydrate thereof.

3. The composition of claim 1, wherein the delivery agent is N-(5-chlorosalicyloyl)-8-aminocaprylic acid or a salt thereof, solvate thereof, or hydrate thereof.

4. The composition of claim 1, wherein the calcitonin is selected from the group consisting of salmon calcitonin, eel calcitonin, human calcitonin, porcine calcitonin, and any combination of any of the foregoing.

5. An oral dosage unit form comprising (a) a delivery agent, (b) parathyroid hormone, (c) calcitonin, and (d) an excipient, a diluent, a disintegrant, a lubricant, a plasticizer, a colorant, a dosing vehicle, or any combination thereof.

6. The oral dosage unit form of claim 5, wherein the dosing vehicle is a liquid selected from the group consisting of water, phosphate buffer, 1,2-propane diol, ethanol, and any combination thereof.

7. The oral dosage unit form of claim 5, wherein the dosage unit form is a tablet, a capsule, a powder, or a liquid.

8. A method for administering parathyroid hormone and calcitonin to an animal in need thereof, the method comprising administering orally to the animal the oral dosage unit form of claim 1.

9. A method of treating osteoporosis comprising administering an effective amount of a delivery agent, parathyroid hormone, and calcitonin.

10. A method for orally administering an effective dose of PTH comprising orally coadministering to a patient in need of PTH an effective amount of a PTH and an effective amount of a calcitonin.

11. A method according to claim 10 wherein the calcitonin is salmon calcitonin.

12. A method of stimulating new bone formation comprising orally administering to a patient in need of new bone formation a therapeutically effective amount of a PTH and a therapeutically effective amount of a calcitonin.

13. A method according claim 12 wherein the calcitonin is salmon calcitonin.

14. A method of treatment or prevention of osteoporosis comprising orally administering to a patient in need of said treatment or prevention a therapeutically effective amount of a PTH and a therapeutically effective amount of a calcitonin.

15. A method according to claim 14 wherein said calcitonin is salmon calcitonin.

16. A composition for oral administration comprising a PTH and a calcitonin.

17. A composition according to claim 16 wherein the PTH is a human form of PTH.

18. A composition according to claim 17 wherein the calcitonin is salmon calcitonin.

19. Use of PTH and calcitonin for the preparation of an orally administrable medicament for the stimulation of bone formation.

20. Use according to claim 19 wherein the PTH is a human form of PTH.

21. Use according to claim 20 wherein the calcitonin is salmon calcitonin.

22. A kit for the stimulation of new bone formation comprising PTH and calcitonin suitable for oral administration together with instructions for the oral administration thereof.

23. A pharmaceutical composition for oral delivery comprising a therapeutically effective amount of a PTH fragment and 5-CNAC, said PTH fragment selected from PTH (1-28) to PTH (1-41).

24. A pharmaceutical composition according to claim 23 wherein the PTH is selected from PTH (1-28), PTH (1-31), PTH (1-34), PTH (1-37), PTH (1-38) and PTH (1-41).

25. A pharmaceutical composition according to claim 23 wherein the PTH is PTH (1-34).

26. A pharmaceutical composition according to claim 23 wherein the PTH is recombinant PTH.

27. A pharmaceutical composition according to claim 26 wherein the PTH is recombinant PTH.

28. A pharmaceutical composition according to claim 23 wherein the PTH is human parathyroid hormone.

29. A pharmaceutical composition according to claim 28 wherein the human parathyroid hormone is hPTH (1-34).

30. A pharmaceutical composition according to claim 23 wherein the 5-CNAC is selected from the free acid, the disodium salt of N-(5-chlorosalicyloyl)-8-aminocaprylic acid and the monohydrate thereof.

31. A pharmaceutical composition according to claim 23 wherein the 5-CNAC is N-(5-chloro-salicyloyl)-8-aminocaprylic acid.

32. A pharmaceutical composition according to claim 23 wherein the 5-CNAC is the disodium salt of N-(5-chloro-salicyloyl)-8-aminocaprylic acid.

33. A method for orally administering an effective dose of PTH comprising orally administering to a patient in need of PTH a pharmaceutical composition comprising a therapeutically effective amount of a PTH fragment and 5-CNAC, said PTH fragment selected from PTH (1-28)-PTH (1-41).

34. A method according to claim 33 wherein the PTH is selected from PTH (1-28), PTH (1-31), PTH (1-34), PTH (1-37), PTH (1-38) and PTH (1-41).

35. A method according to claim 33 wherein the PTH is HPTH (1-34).

36. A method according to claim 33 wherein the PTH is recombinant PTH.

37. A method according to claim 33 wherein the PTH is recombinant hPTH.

38. A method according to claim 33 wherein the 5-CNAC is selected from the free acid, the disodium salt of N-(5-chlorosalicyloyl)-8-aminocaprylic acid and the monohydrate thereof.

39. A method according to claim 33 wherein the 5-CNAC is N-(5-chloro-salicyloyl)-8-aminocaprylic acid.

40. A method according to claim 33 wherein the 5-CNAC is the disodium salt of N-(5-chloro-salicyloyl)-8-aminocaprylic acid.

41. A method of stimulating new bone formation comprising orally administering to a patient in need of new bone formation a pharmaceutical composition comprising a therapeutically effective amount of a PTH fragment and 5-CNAC, said PTH fragment selected from PTH (1-28)-PTH (1-41).

42. A method of treatment or prevention of osteoporosis comprising orally administering to a patient in need of said treatment or prevention a pharmaceutical composition comprising a therapeutically effective amount of a PTH fragment and 5-CNAC, said PTH fragment selected from PTH (1-28)-PTH (1-41).

43. The use of 5-CNAC for the preparation of a pharmaceutical composition suitable for the oral delivery of PTH fragments selected from PTH (1-28)-PTH (1-41).
